(19) United States
(12) Patent Application Publication
(10) Pub. No.: US 2026/0215917 A1
(43) Pub. Date: Jul. 30, 2026

(54) EXPANSION OF PROSTHETIC VALVE DEVICE

(71) Applicant: Pi-Cardia Ltd., Rehovot (IL)

(72) Inventors: Erez Golan, Nes Ziona (IL); Benzion Spector, Be’er Yaakov (IL); Marom Mizrachi, Tel Aviv (IL)

(73) Assignee: Pi-Cardia Ltd., Rehovot (IL)

(21) Appl. No.: 18/997,224

(22) PCT Filed: Aug. 9, 2023
(86) PCT No.: PCT/IB2023/058029

§ 371 Date: Jan. 21, 2025

Related U.S. Application Data
(60) Provisional application No. 63/396,971, filed on Aug. 11, 2022.

(51) Int. Cl. A61F 2/24; A61M 25/00

(52) U.S. Cl. CPC A61F 2/243; A61M 25/0074

(57) ABSTRACT

An expansion device includes expandable arms and a linkage mechanism coupled to the expandable arms for expanding the expandable arms from a contracted position to an expanded position and for contracting the expandable arms back to the contracted position. An outward facing surface of at least one of the expandable arms includes a gripping structure which prevents the at least one of the expandable arms from slipping away from a narrow portion of a prosthetic valve.

### FIELD OF THE INVENTION

The present invention generally relates to prosthetic valve devices and particularly to a device and a method for expanding an under-expanded prosthetic (e.g., transcatheter) valve device.

### BACKGROUND OF THE INVENTION

Prosthetic heart valves are used to replace a native aortic valve, another transcatheter valve, a surgical heart valve or a heart valve that has undergone a surgical procedure. The prosthetic heart valve is delivered to the desired site (e.g., near the native aortic annulus) using any suitable delivery device. During delivery, the prosthetic heart valve is typically disposed inside the delivery device in a collapsed configuration. The delivery device may be introduced into a patient using a transfemoral, transaortic, transsubclavian, transapical, transseptal or any other percutaneous approach. Once the delivery device has reached the target site, the prosthetic heart valve is deployed by expanding it so that the device is secured within the native aortic annulus and native calcified leaflets. When the prosthetic heart valve is properly positioned inside the aortic annulus, it works as a one-way valve, allowing blood to flow from the left ventricle of the heart to the aorta, and preventing blood from flowing in the opposite direction into the ventricle.

A known problem can occur with prosthetic heart valve devices in which the device is under-expanded. For example, in a valve-in-valve procedure, the non-compliant stent of the previous prosthesis may prevent full expansion of the transcatheter aortic valve (TAV), which can result in a distortion of the leaflet configuration, and which can adversely affect durability and functionality of the TAV.

In another example, calcifications in the native valve can prevent full expansion of the TAV.

### SUMMARY OF THE INVENTION

The present invention seeks to provide a device and a method for expanding an under-expanded prosthetic (e.g., transcatheter) valve device, as is explained more in detail hereinbelow.

The expansion device can be used to post-dilate an under-expanded prosthetic valve frame, such as a transcatheter aortic valve replacement (TAVR) frame. The expansion device can be introduced in a suitable percutaneous approach so that the device approaches the TAVR frame above the leaflets. The expansion device may be oriented by rotation and translation so that expandable arms of the device are aligned with the frame dimension that is considered narrow (under-expanded) and which needs dilation. The expandable arms of the device are expanded radially outwards which applies a radially-outward force on the TAVR frame, without endangering the new valve leaflets and without creating any occlusion. The expandable arms expand or press radially outwards directly on the stent frame and outside the leaflets. For example, the expandable arms may extend distally above the leaflets, and contact the frame just above the annulus level, going around the leaflets.

The procedure can be carried out under fluoroscopy and/or echocardiography, carefully controlling the expansion of the arms and thereby carefully controlling the amount of frame deformation (expanding a selected portion of the frame and not necessarily the entire circumference of the frame) so as not to cause any annual rupture or other damage to the leaflets or other tissues. The procedure can reduce or eliminate valve leaflet thickening and reduced motion, referred to as hypo-attenuating leaflet thickening (HALT) or the more severe hypo-attenuation affecting motion (HAM).

The expansion device may include gripping structure on the expandable arms which prevents the arms from slipping away from the narrow portion of the prosthetic valve towards wider portions. This ensures the device expands the prosthetic valve only at the under-expanded portions and not at other portions. The expansion device may include limiting structure that limits the radially-outward expansion of the expandable arms to ensure there is no over-expansion and no annular rupture or other damage.

There is provided in accordance with a non-limiting embodiment of the invention an expansion device including expandable arms and a linkage mechanism coupled to the expandable arms for expanding the expandable arms from a contracted position to an expanded position and for contracting the expandable arms back to the contracted position, wherein an outward facing surface of at least one of the expandable arms includes a gripping structure which prevents the at least one of the expandable arms from slipping away from a narrow portion of a prosthetic valve.

There is provided in accordance with a non-limiting embodiment of the invention a method of expanding an under-expanded portion of a prosthetic valve device, including:
providing an expansion device that includes expandable arms and a linkage mechanism coupled to the expandable arms for expanding the expandable arms from a contracted position to an expanded position and for contracting the expandable arms back to the contracted position,
delivering the expansion device so that the expandable arms are positioned inwards of an under-expanded portion of a prosthetic valve device, and
using the linkage mechanism to expand the expandable arms radially outwards which applies a radially-outward force on the under-expanded portion and corrects expansion of the under-expanded portion.

### BRIEF DESCRIPTION OF THE DRAWINGS

The present invention will be understood and appreciated more fully from the following detailed description, taken in conjunction with the drawings in which:

FIG. 1 is a simplified pictorial illustration of a prosthetic valve device installed in a heart, such as a TAVR which replaces the native aortic valve;

FIG. 2 is a simplified pictorial illustration of an expansion device for expanding the prosthetic valve device, constructed and operative in accordance with a non-limiting embodiment of the invention;

FIG. 3A is a simplified pictorial illustration of the expansion device applying radially-outward expansion forces on the prosthetic valve device;

FIG. 3B is a simplified illustration that shows the radially-outward expansion forces are applied on the narrowest portion of the prosthetic valve device;

FIGS. 4A and 4B are simplified pictorial illustrations of the expansion device applying radially-outward expansion forces on the prosthetic valve device at two different positions (two different implantation depths) of the prosthetic valve device;

FIGS. 5A and 5B are simplified illustrations of the prosthetic valve device before and after expansion, respectively;

FIG. 6 is a simplified pictorial illustration of an expansion device for expanding the prosthetic valve device, constructed and operative in accordance with a non-limiting embodiment of the invention; and

FIG. 7 is a simplified pictorial illustration of the expansion device of FIG. 6 deployed in-situ to apply radially-outward expansion forces on the prosthetic valve device.

### DETAILED DESCRIPTION

Reference is now made to FIG. 1, which illustrates a prosthetic valve device 1 installed in a heart, such as a TAVR which replaces the native aortic valve. The prosthetic valve device 1 has an under-expanded portion 2.

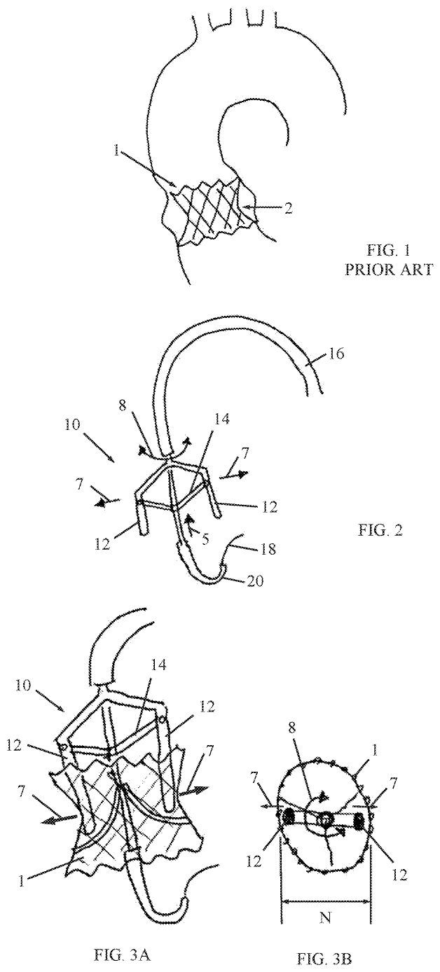

Reference is now made to FIG. 2, which illustrates an expansion device 10 for expanding the prosthetic valve device 1, constructed and operative in accordance with a non-limiting embodiment of the invention.

The expansion device 10 may include expandable arms 12 and a linkage mechanism 14 coupled to the expandable arms 12 for expanding the expandable arms 12 from a contracted position to an expanded position and for contracting the arms back to the contracted position.

The expansion device 10 may be coupled to, or part of, a catheter 16 for delivery over a guidewire 18, as is well known in the art. The expansion device 10 may have a pig-tail end 20, as is known in the art.

In one example, the linkage mechanism 14 can move along the guidewire or other elongate structure, as indicated by arrow 5, to expand the expandable arms 12 to the expanded position, as indicated by arrows 7. The linkage mechanism 14 (or other mechanism) can rotate the expandable arms 12, as indicated by arrow 8, so as to orient the arms 12 in the desired rotational orientation.

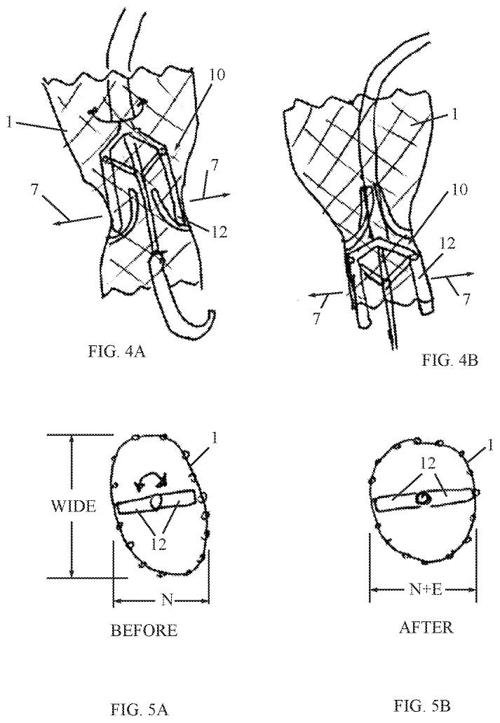

FIG. 3A illustrates the expansion device 10 applying radially-outward expansion forces, indicated by arrows 7, on the prosthetic valve device 1.

FIG. 3B shows the radially-outward expansion forces 7 are applied on the narrowest portion N of the prosthetic valve device 1.

FIGS. 4A and 4B illustrate the expansion device 10 applying radially-outward expansion forces 7 on the prosthetic valve device 1 at two different positions (two different implantation depths) of the prosthetic valve device 1. FIGS. 5A and 5B illustrate the prosthetic valve device 1 before and after expansion, respectively. Before expansion, the prosthetic valve device 1 has an under-expanded narrow dimension N. After expansion, the prosthetic valve device 1 has a corrected dimension N+E.

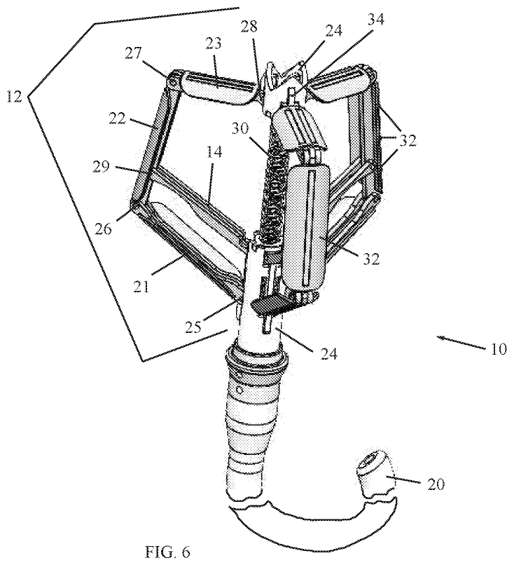

Reference is now made to FIG. 6, which illustrates an example of expansion device 10 in more detail.

The expandable arm 12 may be constructed of two or more hinged members, such as but not limited to, first, second and third hinged members 21, 22 and 23, respectively. First hinged member 21 may be pivotally coupled to a central shaft 24 at a first pivot 25, and to second hinged member 22 at a second pivot 26. Second hinged member 22 may be pivotally coupled to third hinged member 23 at a third pivot 27. Third hinged member 23 may be pivotally coupled to central shaft 24 at a fourth pivot 28.

The linkage mechanism 14 may be coupled to second hinged member 22 at a fifth pivot 29 and may be pivotally coupled to the central shaft 24. The central shaft 24 may include two portions separated by a biasing device 30 such as a spring.

An outward facing surface of one or more of the expandable arms 12 may include gripping structure 32 which prevents the arms 12 from slipping away from the narrow portion of the prosthetic valve towards wider portions. Non-limiting examples of gripping structure 32 include one or more roughened portions (e.g., ridges, knurling, non-smooth texture, and the like) or anti-slip or mildly adhesive coatings. This ensures the device 10 expands the prosthetic valve only at the under-expanded portions and not at other portions.

The expansion device 10 may include limiting structure 34 that limits the radially-outward expansion of the expandable arms 12 to ensure there is no over-expansion and no annular rupture or other damage. A non-limiting example of the limiting structure 34 is an abutment 34 positioned near fourth pivot 28 that prevents third hinged member 23 from moving past it and thus limits the outward expansion of first and second hinged members 21 and 22.

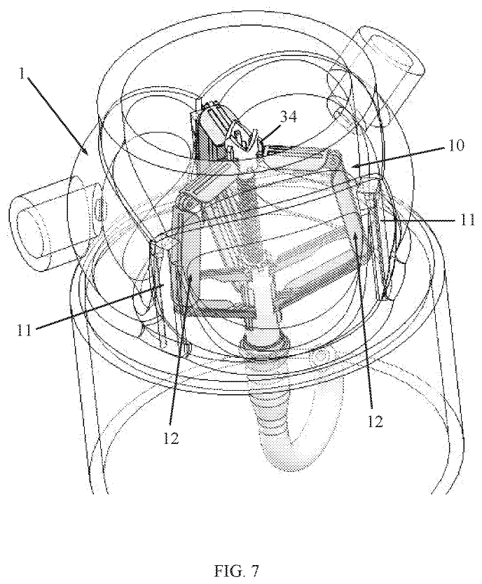

Reference is now made to FIG. 7, which illustrates expansion device 10, deployed in-situ to apply radially-outward expansion forces on the prosthetic valve device 1. It is noted that the expandable arms 12 have been rotationally oriented to press against targeted structural members 11 of prosthetic valve device 1, which have been selected as the members which will receive the corrective expansion forces.

What is claimed is:

1. An expansion device comprising:
expandable arms and a linkage mechanism coupled to said expandable arms for expanding said expandable arms from a contracted position to an expanded position and for contracting said expandable arms back to the contracted position;
said expansion device having a first operative position in which said expandable arms are positioned inwards of an under-expanded portion of a prosthetic valve device, and a second operative position in which said linkage mechanism applies a radially-outward force that expands said expandable arms radially outwards against said under-expanded portion and corrects expansion of said under-expanded portion.

2. The expansion device according to claim 1, wherein an outward facing surface of at least one of said expandable arms comprises a gripping structure which prevents said at least one of said expandable arms from slipping away from a narrow portion or from said under-expanded portion of the prosthetic valve.

3. The expansion device according to claim 2, wherein said gripping structure comprises one or more roughened portions.

4. The expansion device according to claim 2, wherein said gripping structure comprises an anti-slip or adhesive coating.

5. The expansion device according to any one of claims 1-4, further comprising limiting structure that limits radially-outward expansion of said expandable arms.

6. The expansion device according to claim 5, wherein said limiting structure an abutment against which at least one of said expandable arms is abuttable.

7. The expansion device according to any one of claims 1-6, wherein each of said expandable arms is constructed of two or more hinged members.

8. The expansion device according to any one of claims 1-7, wherein said linkage mechanism is pivotally coupled to said expandable arms and pivotally coupled to a central shaft.

9. The expansion device according to any one of claims 1-8, wherein said prosthetic valve device comprises a transcatheter aortic valve replacement (TAVR) device.
